(19) United States
(12) Patent Application Publication
(10) Pub. No.: US 2008/0228167 A1
(43) Pub. Date: Sep. 18, 2008

(54) CATHETER WITH PRESSURE SENSOR SYSTEM

(76) Inventors: Stephan Mittermeyer, Landshut (DE); Andreas Harlep, Holzleinrchen (DE)

Correspondence Address: RENNER, OTTO, BOISSELLE & SKLAR, LLP, Nineteenth Floor, 1621 Euclid Avenue, Cleveland, OH, 44115-2191, US

(21) Appl. No.: 12/048,647

(22) Filed: Mar. 14, 2008

Related U.S. Application Data
(60) Provisional application No. 60/908,216, filed on Mar. 27, 2007.

(30) Foreign Application Priority Data
Mar. 16, 2007 (EP) 07005469.7

(51) Int. Cl. A61M 25/00

(52) U.S. Cl. 604/523

(57) ABSTRACT

A catheter for administering a substance into a patient's tissue including a number of pressure sensors for detecting changes in the shape of the catheter or a backflow along the surface of the catheter. In response to the pressure distribution profile collected along the surface or length of the catheter, the physician may simulate or adapt the substance administration plan to accommodate the actual position of the catheter or the backflow along the surface of the catheter.

### RELATED APPLICATION DATA

This application claims priority of U.S. Provisional Application No. 60/908,216 filed on Mar. 27, 2007, and EP07005469 filed on Mar. 16, 2007, which are incorporated herein by reference in their entirety.

### FIELD OF THE INVENTION

The invention relates to a catheter for administering a substance into a body tissue, in particular into brain structures.

### BACKGROUND OF THE INVENTION

To place a catheter in a patient, a physician ordinarily predetermines or plans a trajectory for the catheter. To assist the physician in adhering to the planned trajectory, the catheter can be made of a relatively rigid material, or the catheter can be used with a stylet made of a rigid material (e.g., stainless steel).

During the placement of the catheter, the physician may encounter obstacles in the form of resistance properties of the anatomical structures in the treatment vicinity or along the planned trajectory. In such circumstances, the placement of some catheters can deviate from the planned trajectory. The deviation can occur due to surfaces present (for example, sulci) or due to heterogeneous properties of the brain tissue (for example, different elasticities). To plan for a reliable and predictable dispersion of a substance in the treatment vicinity, the dispersion may be simulated in advance with computer assistance.

In convection-enhanced delivery, a positive pressure gradient is used to aid in the dispersion of the substance. U.S. Pat. Nos. 5,987,995 and 6,120,457 disclose the use of catheters to measure the pressure at a particular point in the tissue or in the body.

### SUMMARY OF THE INVENTION

A catheter in accordance with the invention is configured for administering a substance into a body tissue (e.g., brain structures), such that the dispersion of the substance introduced through the catheter is reliable and predictable. To this end, the catheter may be configured to change shape while the catheter is being introduced into the tissue and the changes to the shape of the catheter may be detected using pressure sensors integrated into the catheter design. The pressure sensors may also be used to determine the presence and extent of any backflow along an exterior surface of the catheter. The information related to changes in the shape of the catheter or related to the presence of a backflow along the exterior of the catheter may be provided to a computer for simulation and/or adaptation of a treatment plan.

In accordance with one aspect of the invention, a catheter may be provided that includes an elongated catheter body surrounding a lumen (through which a substance can flow) and a pressure sensor array provided on the catheter exterior surface or in the catheter body, wherein the pressure sensor array is used to detect a pressure distribution along a length or portion of the catheter.

In accordance with another aspect of the invention, an end of the catheter that penetrates into the body tissue (the distal end) may include a number of pressure sensors or a pressure sensor system that detects the pressure status of the catheter itself, namely the pressure exerted on the surface material of the catheter or the pressure in the catheter material. In other words, the catheter status is detected by ascertaining a pressure status along the surface of the catheter or within the catheter.

A method in accordance with the invention may include ascertaining the surface or material pressure status of the catheter with the aid of a pressure sensor system, and using the ascertained pressure status to determine the presence or direction of any bends in the catheter or the presence and/or length of any backflow along the exterior of the catheter.

When a pressure distribution over a portion or length of a catheter is detected, it is possible to determine a deformation or change in shape of the catheter. For example, if the catheter is bent or compressed towards one side, a higher pressure in the catheter body material can be measured on an inner side of the catheter (the concave side), and a lower pressure in the catheter body material can be measured on an outer side of the catheter (the convex side). Using information obtained from a catheter equipped with a pressure sensor array, it is possible to ascertain directional information related to the bending of the catheter. From this directional information, the user can obtain real-time feedback related to any deviation from the catheter's planned trajectory. Relying on this feedback, the physician may chose to remove and replace the catheter or adapt an administration plan to accommodate the actual position at which the catheter is administering the substance. The administration plan may be simulated again (with computer assistance) using a new administration point or region, and/or a new administration plan may be developed or simulated with adaptations to the original plan. Such adaptations can be changes to the substance flow rate or changes to the catheter position, such as repositioning the catheter slightly forward or slightly retracted to ensure that the substance is dispersed in the desired manner.

In one embodiment of the invention, the pressure sensor array measures a pressure profile in a catheter body, and is introduced at least partially into the material of the catheter body, e.g., in a catheter wall. Alternatively, the pressure sensor array can be used to measure an ambient pressure profile exerted on the catheter, e.g., the array can be arranged at least partially on a surface of the catheter. The pressure sensor array can be arranged on the surface of the catheter in the form of a coating or covering.

A length or portion of the catheter over which the pressure distribution is detected can be designed in several ways. The length can include an area around a single pressure sensor or an area around a number of pressure sensors.

In accordance with another embodiment of the invention, a number of pressure sensors can be arranged along a substantially longitudinal portion of the catheter. The pressure sensors can be arranged at predetermined locations along the catheter or arranged along the catheter in a uniform or otherwise predetermined spacing. Alternatively or additionally, a number of pressure sensors can be arranged in a substantially cross-sectional plane of the catheter. For example, the sensors can be distributed circumferentially on or in the catheter body. A number of pressure sensors also can be arranged at a distal end of the catheter or over a length at the distal end of the catheter.

The pressure sensors can have various configurations suited to individual treatment cases, and can include piezoelectric elements, resistive wire strain elements (e.g., strain gages), or electrical resistance elements that respond to changes in pressure or length. Interfaces or signal taps and/or signal relaying devices can be provided to relay the data ascertained by the pressure sensors to a computer (for determination of a pressure profile along the catheter's length or for simulating a dispersion in a patient). For example, an embodiment in accordance with the invention can include thin conductive paths that are printed, vapor-deposited or embedded in the catheter material and are arranged with the number of pressure sensors.

If piezoelectric pressure sensors are used, these sensors may be provided as a catheter covering, coating, or as integrated components. In such an arrangement, a measured pressure can be derived from a measured voltage, as the measured voltage at each piezoelectric pressure sensor can be directly or indirectly related to the sensor's degree of bending and/or change in pressure. The measured voltage thus enables the pressure intensity or degree of bending to be quantified.

In accordance with another embodiment of the invention, the catheter configuration can provide continuous feedback concerning a pressure profile along a surface of the catheter body. The pressure profile data obtained can be used as an input variable to deduce the pressure profile in the vicinity of a catheter tip. The pressure profile at the catheter tip may allow the determination of fluid backflow length, as can occur during infusions. The pressure profile data obtained may allow precise simulation of the actual substance dispersion, even while administering the substance. Additionally, pressure profiles observed during the infusion can be used as valuable input variables for further infusion simulations. Treatment after the catheter has been placed may be optimized by altering administering parameters and/or treatment parameters such as the flow rate, administration duration, etc. The observed results of an infusion and the results of any simulations performed may be used in the treatment optimization process.

### BRIEF DESCRIPTION OF THE DRAWINGS

The forgoing and other features of the invention are hereinafter discussed with reference to the figures.

FIG. 1 is a schematic representation of an exemplary catheter in accordance with the invention.

FIG. 2 is a section view of the plane A-A in FIG. 1.

FIG. 3 is a schematic representation of detecting the pressures in the catheter shown in FIGS. 1 and 2.

FIG. 4 is a view of another exemplary catheter in accordance with the invention.

FIG. 5 is an enlarged representation of a detail from FIG. 4.

### DETAILED DESCRIPTION

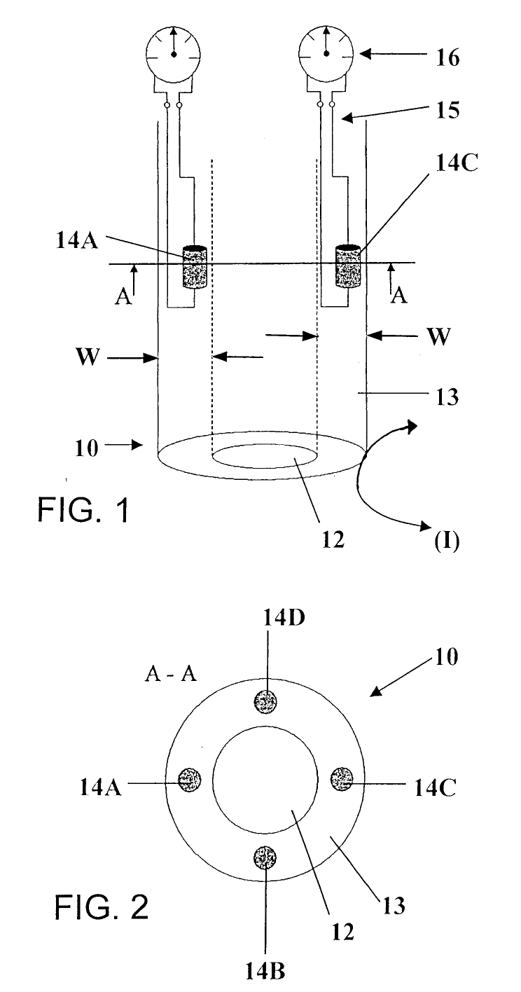

A schematic representation of an exemplary catheter in accordance with the invention is shown in FIG. 1 and FIG. 2, wherein FIG. 2 is a sectional representation on the plane A-A in FIG. 1. The catheter 10 includes a catheter body 13 and a lumen 12 that is enclosed by the catheter body 13 and through which a substance, for example a drug, is infused into a body tissue.

In this embodiment, a plurality of pressure sensors 14A through 14D are introduced in the walls W of the catheter body 13, and the pressure sensors 14A and 14C can be seen in the representation in FIG. 1. The pressure sensors 14A and 14C schematically represent sensors (e.g., piezoelectric crystal elements) that, when deformed (e.g., in the longitudinal axis, not shown), emit electrical signals, such as voltage or current signals. These voltage are tapped or provided at the pressure sensors 14A-14D and conducted by a plurality of electrical conductive paths 15 (e.g., printed conductive paths or thin metal fibers) to a measuring device 16 in FIG. 1.

In FIG. 2, four pressure sensors 14A, 14B, 14C, and 14D are shown in an array, wherein the sensors are distributed about a circumference of the catheter body or lumen (in this example they are equally spaced at 90° intervals).

When being placed into a brain tissue, the catheter 10 may be bent in a direction (I). In a bent condition, the pressure in the catheter material on the side of the sensor 14C will increase due to material compression. Conversely, the pressure in the catheter material on the side of the sensor 14A will decrease due to material expansion.

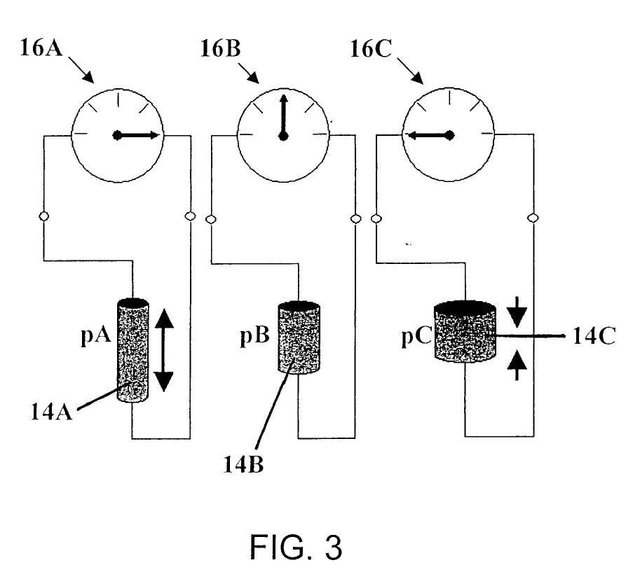

FIG. 3 schematically illustrates three sensors 14A, 14B, and 14C after the catheter 10 is bent in the direction (I). The pressure sensor 14A (located on the expansion side of the catheter) measures a pressure PA that is lower than a pressure PB at the pressure sensor 14B (located at a neutral side or zone of the catheter). A pressure PC measured by the pressure sensor 14C (located on the compression side of the catheter) is correspondingly higher than the pressure PB measured by the pressure sensor 14B. The corresponding pressure measuring devices 16A, 16B, and 16C report these pressure measurements to a physician or provide these measurements to a computer (not shown) for further processing. The measurements of the pressures PA, PB, PC (or pressure profile) can be voltage or current values, for example, provided or tapped at each respective pressure sensor 14A, 14B, 14C (e.g., piezoelectric pressure measuring device) and provided to the computer to qualitatively determine that the catheter has been bent in the direction of the arrow (I), and quantitatively determine the extent of the bending.

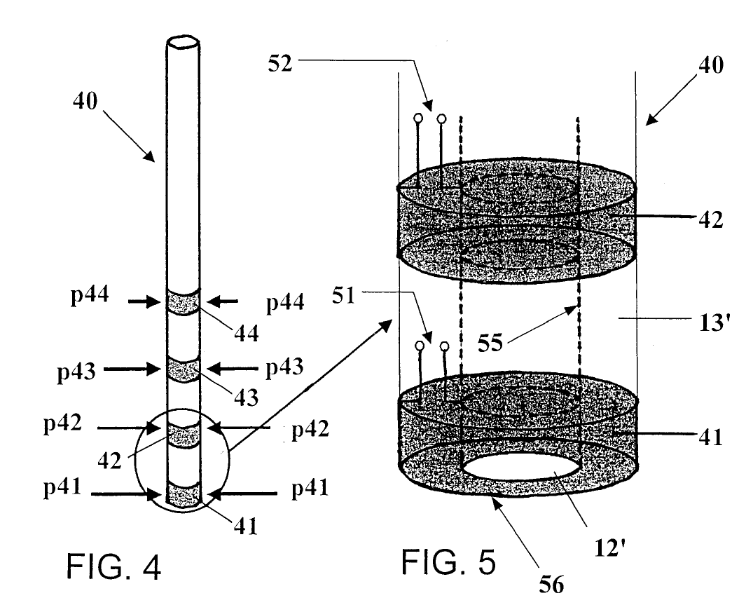

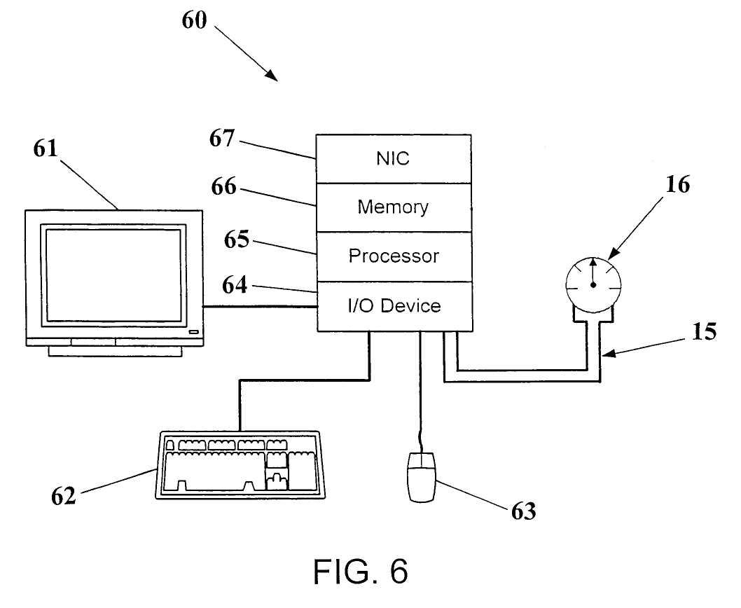

FIG. 4 illustrates another exemplary catheter 40 in accordance with the invention, wherein the lower detail in FIG. 4 is shown in an enlargement in FIG. 5 (the catheter body is 13′ and the lumen is 12′). The catheter 40 of FIG. 4 has annular pressure sensors 41, 42, 43, and 44 that are arranged on the distal portion of the catheter 40 at known locations having predetermined distances from each other. The pressure sensors 41, 42, 43, and 44 indicate respective pressure ratios or differences (comparison of internal and external pressure measurements) in their respective catheter portions or locations. The pressure sensors 41, 42, 43, and 44 may include signal or measurement relaying devices 51 and 52 operably connected to the pressure sensors 41 and 42 in FIG. 5. The pressure sensors can again detect bending, however, in this embodiment the pressure sensors also may measure and report a pressure profile over the length of the catheter 40. Such a pressure profile is schematically illustrated in FIG. 4 by the pressure arrows P41, P42, P43, and P44, wherein the pressures are lower or higher in accordance with the length of the arrows.

When such a catheter 40 is used to administer a liquid drug into a portion of a patient's body (e.g., into a brain tissue), a so-called backflow is generated (e.g., the drug returns along the exterior of the catheter). When a backflow is generated, the drug can exert a pressure on a catheter wall 55, and this pressure decreases with the distance from a catheter tip 56, such that the pressures P41 to P44 decrease from the sensor 41 to the sensor 44. Such a pressure distribution can be measured using the sensors 41 to 44, and the distribution can provide the user information concerning the actual backflow status, in particular how far along the catheter 40 (from the catheter tip 56 towards the proximal end) the backflow region extends. This information can be used in a new simulation of the dispersion of the drug or to adapt and/or improve an already existing simulation. Should the simulation reveal that treatment adaptations or adjustments are desired due to the actual backflow (e.g., adaptations to the flow rate and/or duration of infusion), such adaptations can be made to ensure a positive treatment result.

Various embodiments of pressure sensors have been shown in the figures (pressure sensors in the catheter wall in FIG. 1; pressure sensors such as annular sensors in FIGS. 4 and 5), however the invention is not limited to such types of pressure sensors. Rather, catheters in accordance with the invention also can include pressure sensors arranged as coverings or coatings on the outside of the catheter circumference, or on the inside of the surface of the catheter wall which surrounds the lumen.

Moving now to FIG. 6 there is shown a block diagram of an exemplary data processing device or computer 60 that may be used to implement one or more of the methods described herein. The computer 60 may include a display 61 for viewing system information, and a keyboard 62 and pointing device 63 for data entry, screen navigation, etc. A computer mouse or other device that points to or otherwise identifies a location, action, etc., e.g., by a point and click method or some other method, are examples of a pointing device 63. Alternatively, a touch screen (not shown) may be used in place of the keyboard 62 and pointing device 63. The display 61, keyboard 62 and mouse 63 communicate with a processor via an input/output device 64, such as a video card and/or serial port (e.g., a USB port or the like).

A processor 65, such as an AMD Athlon 64® processor or an Intel Pentium IV® processor, combined with a memory 66 execute programs to perform various functions, such as data entry, numerical calculations, screen display, system setup, etc. The memory 66 may comprise several devices, including volatile and non-volatile memory components. Accordingly, the memory 66 may include, for example, random access memory (RAM), read-only memory (ROM), hard disks, floppy disks, optical disks (e.g., CDs and DVDs), tapes, flash devices and/or other memory components, plus associated drives, players and/or readers for the memory devices. The processor 65 and the memory 66 are coupled using a local interface (not shown). The local interface may be, for example, a data bus with accompanying control bus, a network, or other subsystem.

The memory may form part of a storage medium for storing information, such as application data, screen information, programs, etc., part of which may be in the form of a database. The storage medium may be a hard drive, for example, or any other storage means that can retain data, including other magnetic and/or optical storage devices. A network interface card (NIC) 67 allows the computer 60 to communicate with other devices such as the plurality of electrical conductive paths 15 (e.g., printed conductive paths or thin metal fibres) or the measuring device 16.

A person having ordinary skill in the art of computer programming and applications of programming for computer systems would be able in view of the description provided herein to program a computer system 60 to operate and to carry out the functions described herein. Accordingly, details as to the specific programming code have been omitted for the sake of brevity. Also, while software in the memory 66 or in some other memory of the computer and/or server may be used to allow the system to carry out the functions and features described herein in accordance with the preferred embodiment of the invention, such functions and features also could be carried out via dedicated hardware, firmware, software, or combinations thereof, without departing from the scope of the invention.

Computer program elements of the invention may be embodied in hardware and/or in software (including firmware, resident software, micro-code, etc.). The invention may take the form of a computer program product, which can be embodied by a computer-usable or computer-readable storage medium having computer-usable or computer-readable program instructions, “code” or a “computer program” embodied in the medium for use by or in connection with the instruction execution system. In the context of this document, a computer-usable or computer-readable medium may be any medium that can contain, store, communicate, propagate, or transport the program for use by or in connection with the instruction execution system, apparatus, or device. The computer-usable or computer-readable medium may be, for example but not limited to, an electronic, magnetic, optical, electromagnetic, infrared, or semiconductor system, apparatus, device, or propagation medium such as the Internet. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner. The computer program product and any software and hardware described herein form the various means for carrying out the functions of the invention in the example embodiments.

Although the invention has been shown and described with respect to a certain preferred embodiment or embodiments, it is obvious that equivalent alterations and modifications will occur to others skilled in the art upon the reading and understanding of this specification and the annexed Figures. In particular regard to the various functions performed by the above described elements (components, assemblies, devices, software, computer programs, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A catheter for administering a substance into a patient's tissue, comprising:
an elongated catheter body that surrounds a lumen; and
a plurality of pressure sensors provided in or on the catheter body,
wherein the plurality of pressure sensors detect a pressure distribution over a portion of the catheter body.

2. The catheter according to claim 1, wherein the plurality of pressure sensors are positioned at least partially within a material that comprises the catheter body and are operable to measure a pressure profile in or on the material.

3. The catheter according to claim 1, wherein the plurality of pressure sensors are positioned at least partially on a surface of the catheter body and are operable to measure an ambient pressure profile exerted on the surface of the catheter body.

4. The catheter according to claim 1, wherein the plurality of pressure sensors are arranged on a surface of the catheter body in the form of a coating or covering.

5. The catheter according to claim 1, wherein the plurality of pressure sensors are arranged in a cross-sectional plane of the catheter and are distributed circumferentially on or in the catheter body.

6. The catheter according to claim 1, wherein the plurality of pressure sensors are arranged along a longitudinal portion of the catheter body at predetermined locations and/or in a uniform spacing.

7. The catheter according to claim 1, wherein the plurality of pressure sensors are arranged at a distal end (away from a handle end) of the catheter or over a length at the distal end of the catheter.

8. The catheter according to claim 1, wherein the plurality of pressure sensors include piezoelectric elements.

9. The catheter according to claim 1, wherein the plurality of pressure sensors include resistive wire strain elements.

10. The catheter according to claim 1, wherein the plurality of pressure sensors include electrical resistance elements that respond to changes in pressure or length.

11. The catheter according to claim 1, further comprising a plurality of interfaces and/or signal taps or signal relaying devices arranged on the plurality of pressure sensors.

12. A method for administering a substance into a patient's tissue, comprising:
providing a catheter having an elongated catheter body that surrounds a lumen, said body including a plurality of pressure sensors,
detecting a pressure distribution over a portion of the catheter via the plurality of pressure sensors, and
determining a change in shape of the catheter based on the detected pressure distribution.

13. The method according to claim 12, further comprising determining the extent and direction of the change in shape of the catheter.

14. The method according to claim 12, further comprising simulating the dispersion of a substance from the catheter, said simulation performed with computer assistance.

15. The method according claim 12, further comprising preparing a substance administration plan and applying an adaptation to the substance administration plan based on the change in shape of the catheter.

16. The method according to claim 15, wherein the adaptation to the substance administration plan comprises changes to a substance flow rate or a change to the catheter position.

17. A method for administering a substance into a patient's tissue, comprising:
providing a catheter having an elongated catheter body that surrounds a lumen, said body including a plurality of pressure sensors,
detecting a pressure distribution over a portion of the catheter via the plurality of pressure sensors, and
determining the presence and/or extent of a backflow along the catheter body based on the detected pressure distribution.

18. The method of claim 17, further comprising simulating the dispersion of a substance from the catheter with computer assistance, in the advance of or during treatment.

19. The method of claim 17, further comprising using the detected pressure distribution in a real-time simulation of the dispersion of a substance from the catheter.

20. The method of claim 17, further comprising: preparing a substance administration plan; and applying an adaptation to the substance administration plan based on the presence and extent of the backflow.
